(19) United States
(12) Patent Application Publication
(10) Pub. No.: US 2025/0273322 A1
(43) Pub. Date: Aug. 28, 2025

(54) MEDICAL IMAGE ANALYSIS METHOD BASED ON DEEP LEARNING

(71) Applicant: VUNO Inc., Seoul (KR)

(72) Inventors: Kyungdoc KIM, Seoul (KR); Jeonghyuk PARK, Incheon (KR); Hongseok LEE, Seoul (KR); Chanmin PARK, Seoul (KR)

(21) Appl. No.: 18/702,523

(22) PCT Filed: Oct. 19, 2022
(86) PCT No.: PCT/KR2022/015911

§ 371 Date: Apr. 18, 2024

(30) Foreign Application Priority Data
Oct. 20, 2021 (KR) 10-2021-0139897

(51) Int. Cl. G16H 30/40; G06T 7/00

(52) U.S. Cl. CPC G16H 30/40; G06T 7/0012; G06T 2207/20084; G06T 2207/30024

(57) ABSTRACT

Disclosed is a method for analyzing a medical image based on deep learning, which is performed by a computing device. The method may include: obtaining position information of stained cells present in a medical image by using a pre-trained neural network model; and calculating a staining ratio of the stained cells in a bounding box including the stained cells corresponding to the position information.

### TECHNICAL FIELD

The present disclosure relates to a method for analyzing a medical image, and more particularly, to a method for performing analysis on a stained cell present in a medical image by using artificial intelligence.

### DESCRIPTION OF THE ART

Conventional pathological diagnostic tests involve making cell or tissue samples collected from the human body as glass slides and examining the cell or tissue samples with a microscope. Since the conventional testing method detects and classifies each cell on the slide with the naked eyes, a testing speed is slow and it inevitably takes a long time to confirm a final test result. As a result, the conventional testing methods cause delays in diagnosis and treatment of patients. In particular, in the current era where the number of pathological diagnoses has rapidly increased due to the aging population and increase in cancer patients, the conventional testing methods are no longer considered appropriate. Therefore, the need for digital pathology has been increasing recently.

The digital pathology refers to a method of acquiring digital images from glass slides using a scanner and managing, sharing, and analyzing the digital images in a computing environment, pathological diagnosis rather than a conventional method of examining glass slides with the naked eye through a microscope. The digital pathology provides an environment in which pathological diagnosis work can be performed efficiently by automatically analyzing the digital images of the glass slides in the computing environment. In other words, the digital pathology improves the problem of delay in test, which is a problem of the conventional testing methods, and provides an environment in which patient diagnosis and treatment can be carried out efficiently.

Korean Patent Unexamined Publication No. 10-2020-0117222 (Oct. 14, 2020) discloses an apparatus and a method for supporting a pathological diagnosis.

### BRIEF SUMMARY

### Technical Problem

The present disclosure is contrived in response to the background art, and has been made in an effort to provide a method for analyzing stained cells present in a medical image based on deep learning.

### Technical Solution

According to an embodiment of the present disclosure for achieving the object, disclosed is a method for analyzing a medical image based on deep learning, which is performed by a computing device. The method may include: obtaining position information of stained cells present in a medical image by using a pre-trained neural network model; and calculating a staining ratio of the stained cells in a bounding box including the stained cells corresponding to the position information.

In an alternative embodiment, the obtaining of the position information of the stained cells present in the medical image may include obtaining the bounding box including the stained cells and coordinate values of the bounding box by inputting the medical image into the neural network model.

In an alternative embodiment, the neural network model may be pre-trained based on whether a cell expressed by staining is positive or negative and a medical image in which the bounding box including the cell is labeled.

In an alternative embodiment, the staining ratio of the stained cells may be a ratio of an area of a positive region of cells expressed by staining in the bounding box to a total area of the bounding box.

In an alternative embodiment, the calculating of the staining ratio of the stained cells may include generating a binary image for the bounding box based on a staining intensity of the medical image, and calculating the staining ratio of the stained cells based on the binary image.

In an alternative embodiment, the binary image may be generated based on a result of comparing the staining intensity of the bounding box and a first threshold. At this time, first threshold may be a staining intensity which becomes a reference for classifying the stained cells into positive cells.

In an alternative embodiment, the calculating of the staining ratio of the stained cells based on the binary image may include extracting a reference region from the binary image based on a result of comparing a size of the binary image and a second threshold, and calculating the staining ratio of the stained cells based on the extracted reference region.

In an alternative embodiment, when the size of the binary image is equal to or less than the second threshold, the reference region may be a whole region of the binary image.

In an alternative embodiment, when the size of the binary image is more than the second threshold, the reference region may be a partial region of the binary image based on a center of the binary image.

In an alternative embodiment, the method may further include transmitting, to a user terminal, the position information of the stained cells obtained through the neural network model and the calculated staining ratio. At this time, the position information may include the coordinate values of the bounding box of the medical image corresponding to the position information. Further, the staining ratio may be a value of a ratio of an area of a positive region of cells expressed by staining in the bounding box of the medical image corresponding to the position information to a total area of the bounding box

According to an embodiment of the present disclosure for achieving the object, disclosed is a computer program stored in a computer-readable storage medium. When the computing program is executed by one or more processors, the computer program may allow the one or more processors to execute the following operations for analyzing a medical image based on deep learning, and the operation may include: an operation of obtaining position information of stained cells present in a medical image by using a pre-trained neural network model; and an operation of calculating a staining ratio of the stained cells in a bounding box including the stained cells corresponding to the position information.

According to an embodiment of the present disclosure for achieving the object, disclosed is a computing device for analyzing a medical image based on deep learning. The device may include: a processor including at least one core; a memory including program codes executable in the processor; and a network unit receiving a medical image including a chest region, and the processor may be configured to obtain position information of stained cells present in a medical image by using a pre-trained neural network model, and calculate a staining ratio of the stained cells in a bounding box including the stained cells corresponding to the position information.

### Advantageous Effects

According to the present disclosure, a method for analyzing stained cells present in a medical image based on deep learning, and counting the number of stained cells can be provided.

### BRIEF DESCRIPTION OF THE SEVERAL VIEWS OF THE DRAWINGS

FIG. 1 is a block diagram of a computing device for analyzing a medical image according to an embodiment of the present disclosure.

FIG. 2 is a schematic view illustrating a neural network according to an embodiment of the present disclosure.

FIG. 3 is a block diagram illustrating a process of analyzing the medical image of the computing device according to an embodiment of the present disclosure.

FIG. 4 is a conceptual view ‘illustrating’? of a process of generating a binary image by the computing device according to an embodiment of the present disclosure.

FIG. 5 is a flowchart illustrating a process of analyzing the medical image of the computing device according to an embodiment of the present disclosure.

FIG. 6 is a schematic view of a computing environment according to an embodiment of the present disclosure.

### DETAILED DESCRIPTION

Various exemplary embodiments will now be described with reference to drawings. In the present specification, various descriptions are presented to provide appreciation of the present disclosure. However, it is apparent that the exemplary embodiments can be executed without the specific description.

“Component,” “module,” “system,” and the like which are terms used in the specification refer to a computer-related entity, hardware, firmware, software, and a combination of the software and the hardware, or execution of the software. For example, the component may be a processing procedure executed on a processor, the processor, an object, an execution thread, a program, and/or a computer, but is not limited thereto. For example, both an application executed in a computing device and the computing device may be the components. One or more components may reside within the processor and/or a thread of execution. One component may be localized in one computer. One component may be distributed between two or more computers. Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

The term “or” is intended to mean not exclusive “or” but inclusive “or.” That is, when not separately specified or not clear in terms of a context, a sentence “X uses A or B” is intended to mean one of the natural inclusive substitutions. That is, the sentence “X uses A or B” may be applied to any of the case where X uses A, the case where X uses B, or the case where X uses both A and B. Further, it should be understood that the term “and/or” used in this specification designates and includes all available combinations of one or more items among enumerated related items.

It should be appreciated that the term “comprise” and/or “comprising” means presence of corresponding features and/or components. However, it should be appreciated that the term “comprises” and/or “comprising” means that presence or addition of one or more other features, components, and/or a group thereof is not excluded. Further, when not separately specified or it is not clear in terms of the context that a singular form is indicated, it should be construed that the singular form generally means “one or more” in this specification and the claims.

The term “at least one of A or B” should be interpreted to mean “a case including only A,” “a case including only B,” and “a case in which A and B are combined.”

Those skilled in the art need to recognize that various illustrative logical blocks, configurations, modules, circuits, means, logic, and algorithm steps described in connection with the exemplary embodiments disclosed herein may be additionally implemented as electronic hardware, computer software, or combinations of both sides. To clearly illustrate the interchangeability of hardware and software, various illustrative components, blocks, configurations, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented exemplary embodiments is provided so that those skilled in the art of the present disclosure use or implement the present disclosure. Various modifications to the exemplary embodiments will be apparent to those skilled in the art. Generic principles defined herein may be applied to other embodiments without departing from the scope of the present disclosure. Therefore, the present disclosure is not limited to the exemplary embodiments presented herein. The present disclosure should be analyzed within the widest range which is coherent with the principles and new features presented herein.

Meanwhile, the term “image” or “video” used throughout the detailed description and claims of the present disclosure refers to multi-dimensional data constituted by discrete image elements (e.g., pixels in a 2D image), and in other words, refers to an object which may be seen with an eye (e.g., displayed on a video screen) or a digital representation of the object (such as a file corresponding to a pixel output of CT, MRI detector, etc.).

For example, the “image” or “video” may be a medical image of a subject collected by computed tomography (CT), magnetic resonance imaging (MRI), ultrasonic waves, pathology scan, or any other medical imaging system known in the technical field of the present disclosure.

Throughout the detailed description and claims of the present disclosure, a ‘Digital Imaging and Communications in Medicine (DICOM)’ standard is a term which collectively refers to several standards used for digital image representation and communication in a medical device, so that the DICOM standard is announced by the Federation Committee, constituted in the American College Radiology (ACR) and the National Electrical Manufacturers Association (NEMA).

Further, throughout the detailed description and claims of the present disclosure, a ‘Picture Archiving and Communication System (PACS)’ is a term that refers to a system for performing storing, processing, and transmitting according to the DICOM standard, and medical images acquired by using digital medical image equipment such as X-ray, CT, MRI, and pathology scanner may be stored in a DICOM format and transmitted to terminals inside or outside a hospital through a network, and additionally include a reading result and a medical chart.

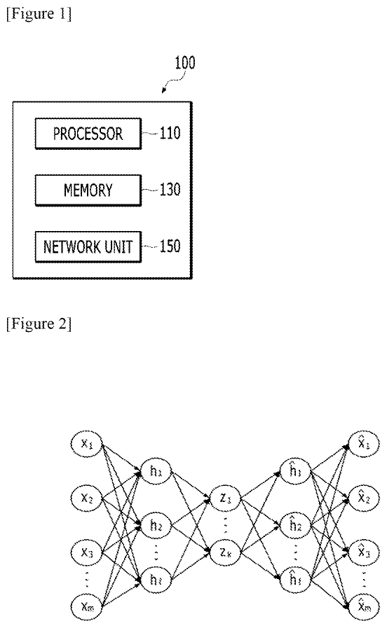

FIG. 1 is a block diagram of a computing device for analyzing a medical image according to an embodiment of the present disclosure.

A configuration of the computing device 100 illustrated in FIG. 1 is only an example shown through simplification. In an exemplary embodiment of the present disclosure, the computing device 100 may include other components for performing a computing environment of the computing device 100 and only some of the disclosed components may constitute the computing device 100.

The computing device 100 may include a processor 110, a memory 130, and a network unit 150.

The processor 110 may be constituted by one or more cores and may include processors for data analysis and deep learning, which include a central processing unit (CPU), a general purpose graphics processing unit (GPGPU), a tensor processing unit (TPU), and the like of the computing device. The processor 110 may read a computer program stored in the memory 130 to perform data processing for machine learning according to an exemplary embodiment of the present disclosure. According to an exemplary embodiment of the present disclosure, the processor 110 may perform a calculation for learning the neural network. The processor 110 may perform calculations for learning the neural network, which include processing of input data for learning in deep learning (DL), extracting a feature in the input data, calculating an error, updating a weight of the neural network using backpropagation, and the like. At least one of the CPU, GPGPU, and TPU of the processor 110 may process learning of a network function. For example, both the CPU and the GPGPU may process the learning of the network function and data classification using the network function. Further, in an exemplary embodiment of the present disclosure, processors of a plurality of computing devices may be used together to process the learning of the network function and the data classification using the network function. Further, the computer program executed in the computing device according to an exemplary embodiment of the present disclosure may be a CPU, GPGPU, or TPU executable program.

According to an embodiment of the present disclosure, the processor 110 may analyze information on tissues or cells present in a medical image. The processor 110 inputs the medical image into a neural network model to analyze the tissues or cells present in the medical image. The processor 110 may generate information required for pathological diagnosis based on information on the tissues or cells output through the neural network model. For example, the processor 110 may estimate a position of the stained cell in the medical image including the stained cells through immunohistochemistry by using the neural network model. The processor 110 may also identify structures such as cell nucleus, cell membrane, cytoplasm, etc., of the stained cell present in the medical image by using the neural network model. The processor 110 may classify the stained cells present in the medical image according to a staining color, a staining intensity, a size of the stained cell, a pattern of the stained cell, a shape of the stained cell, etc., based on information on a position and a structure of a cell estimated through the neural network model. The processor 110 counts the classified stained cells to generate a numerical value corresponding to a counting result. The numerical value generated through the processor 110 may be utilized for pathology diagnosis based on the stained cells.

Such an operation of the processor 110 enables information on the number of stained cells, which is information required for the pathology diagnosis to be quickly and accurately generated. That is, the processor 110 processes the number of stained cells, which conventionally had to be identified with human naked eyes in a computing environment based on an artificial neural network to effectively improve a problem of a conventional pathology test method which is highly dependent on a person's subjective judgment and perception.

According to an exemplary embodiment of the present disclosure, the memory 130 may store any type of information generated or determined by the processor 110 and any type of information received by the network unit 150.

According to an exemplary embodiment of the present disclosure, the memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The description of the memory is just an example and the present disclosure is not limited thereto.

The network unit 150 according to an exemplary embodiment of the present disclosure may use an arbitrary type known wired/wireless communication systems.

The network unit 150 may receive a medical image in which a tissue or cell is expressed from a medical image storage and transmission system. The medical image in which the tissues or cells are expressed may be training data or inference data of the neural network model. For example, the medical image in which the tissue or cell is expressed may be a pathology slide image including at least one tissue or cell. At this time, the pathology slide image may be understood as a scan image acquired from a glass slide through a scanner for pathology diagnosis and stored in the medical image storage and transmission system. The tissue or cell expressed in the pathology slide image may be a tissue or cell stained through the immunohistochemistry.

The immunohistochemistry refers to a method for detecting a target protein or antigen in the tissue. Specifically, the immunohistochemistry refers to a method of exposing a labeled antibody that can bind to the epitope of the target protein or antigen to a tissue section, and visualizing the labeled antibody through tissue staining. For example, the immunohistochemistry may be performed by performing an antibody reaction against a cancer cell proliferation marker such as ki-67 on a glass slide, and then visualizing, through staining, a position where ki-67 is expressed using a specific solution such as Diaminobenzidine (DAB). Cells stained by using the DAB solution may be marked with a brown color in the pathology slide image. At this time, the degree of ki-67 expression in the stained cells may be distinguished depending on the intensity of the brown color. Since the ki-67 and the DAB solution are only examples used in the immunohistochemistry, the immunohistochemistry described in the present disclosure is not limited to the above-described examples.

In addition, the network unit 150 may transmit and receive information processed by the processor 110, a user interface, and the like through communication with other terminals. For example, the network unit 150 may provide the user interface generated by the processor 110 to a client (e.g., a user terminal). In addition, the network unit 150 may receive an external input of a user applied to a client and transfer the external input to the processor 110. In this case, the processor 110 may process operations such as outputting, correcting, changing, adding, and the like of information provided through the user interface based on the external input of the user received from the network unit 150.

Meanwhile, according to an embodiment of the present disclosure, the computing device 100 may include a server as a computing system that transmits and receives information through communication with the client. In this case, the client may be any type of terminal which may access the server. For example, the computing device 100 as the server may receive the medical image from the medical image photographing system or the user terminal and count the number of cells, and provide analysis information including a counting result to the user terminal. At this time, the user terminal may output the user interface received from the computing device 100 as the server, and receive or process information through interaction with the user.

In an additional embodiment, the computing device 100 may also include any type of terminal that receives data resources generated by an arbitrary server and performs additional information processing.

FIG. 2 is a schematic view illustrating a neural network according to an embodiment of the present disclosure.

The neural network model according to an embodiment of the present disclosure may include a neural network for estimating a position of a stained tissue or cell present in the medical image. The neural network may be generally constituted by an aggregate of calculation units which are mutually connected to each other, which may be called nodes. The nodes may also be called neurons. The neural network is configured to include one or more nodes. The nodes (alternatively, neurons) constituting the neural networks may be connected to each other by one or more links.

In the neural network, one or more nodes connected through the link may relatively form the relationship between an input node and an output node. Concepts of the input node and the output node are relative and a predetermined node which has the output node relationship with respect to one node may have the input node relationship in the relationship with another node and vice versa. As described above, the relationship of the input node to the output node may be generated based on the link. One or more output nodes may be connected to one input node through the link and vice versa.

In the relationship of the input node and the output node connected through one link, a value of data of the output node may be determined based on data input in the input node. Here, a link connecting the input node and the output node to each other may have a weight. The weight may be variable and the weight is variable by a user or an algorithm in order for the neural network to perform a desired function. For example, when one or more input nodes are mutually connected to one output node by the respective links, the output node may determine an output node value based on values input in the input nodes connected with the output node and the weights set in the links corresponding to the respective input nodes.

As described above, in the neural network, one or more nodes are connected to each other through one or more links to form a relationship of the input node and output node in the neural network. A characteristic of the neural network may be determined according to the number of nodes, the number of links, correlations between the nodes and the links, and values of the weights granted to the respective links in the neural network. For example, when the same number of nodes and links exist and there are two neural networks in which the weight values of the links are different from each other, it may be recognized that two neural networks are different from each other.

The neural network may be constituted by a set of one or more nodes. A subset of the nodes constituting the neural network may constitute a layer. Some of the nodes constituting the neural network may constitute one layer based on the distances from the initial input node. For example, a set of nodes of which distance from the initial input node is n may constitute n layers. The distance from the initial input node may be defined by the minimum number of links which should be passed through for reaching the corresponding node from the initial input node. However, a definition of the layer is predetermined for description and the order of the layer in the neural network may be defined by a method different from the aforementioned method. For example, the layers of the nodes may be defined by the distance from a final output node.

The initial input node may mean one or more nodes in which data is directly input without passing through the links in the relationships with other nodes among the nodes in the neural network. Alternatively, in the neural network, in the relationship between the nodes based on the link, the initial input node may mean nodes which do not have other input nodes connected through the links. Similarly thereto, the final output node may mean one or more nodes which do not have the output node in the relationship with other nodes among the nodes in the neural network. Further, a hidden node may mean nodes constituting the neural network other than the initial input node and the final output node.

In the neural network according to an exemplary embodiment of the present disclosure, the number of nodes of the input layer may be the same as the number of nodes of the output layer, and the neural network may be a neural network of a type in which the number of nodes decreases and then, increases again from the input layer to the hidden layer. Further, in the neural network according to another exemplary embodiment of the present disclosure, the number of nodes of the input layer may be smaller than the number of nodes of the output layer, and the neural network may be a neural network of a type in which the number of nodes decreases from the input layer to the hidden layer. Further, in the neural network according to yet another exemplary embodiment of the present disclosure, the number of nodes of the input layer may be larger than the number of nodes of the output layer, and the neural network may be a neural network of a type in which the number of nodes increases from the input layer to the hidden layer. The neural network according to still yet another exemplary embodiment of the present disclosure may be a neural network of a type in which the neural networks are combined.

A deep neural network (DNN) may refer to a neural network that includes a plurality of hidden layers in addition to the input and output layers. When the deep neural network is used, the latent structures of data may be determined. That is, latent structures of photos, text, video, voice, and music (e.g., what objects are in the photo, what the content and feelings of the text are, what the content and feelings of the voice are) may be determined. The deep neural network may include a convolutional neural network (CNN), a recurrent neural network (RNN), an auto encoder, generative adversarial networks (GAN), a restricted Boltzmann machine (RBM), a deep belief network (DBN), a Q network, a U network, a Siam network, a Generative Adversarial Network (GAN), and the like. The description of the deep neural network described above is just an example and the present disclosure is not limited thereto.

In an exemplary embodiment of the present disclosure, the network function may include the auto encoder. The auto encoder may be a kind of artificial neural network for outputting output data similar to input data. The auto encoder may include at least one hidden layer and odd hidden layers may be disposed between the input and output layers. The number of nodes in each layer may be reduced from the number of nodes in the input layer to an intermediate layer called a bottleneck layer (encoding), and then expanded symmetrical to reduction to the output layer (symmetrical to the input layer) in the bottleneck layer. The auto encoder may perform non-linear dimensional reduction. The number of input and output layers may correspond to a dimension after preprocessing the input data. The auto encoder structure may have a structure in which the number of nodes in the hidden layer included in the encoder decreases as a distance from the input layer increases. When the number of nodes in the bottleneck layer (a layer having a smallest number of nodes positioned between an encoder and a decoder) is too small, a sufficient amount of information may not be delivered, and as a result, the number of nodes in the bottleneck layer may be maintained to be a specific number or more (e.g., half of the input layers or more).

The neural network may be learned in at least one scheme of supervised learning, unsupervised learning, semi supervised learning, or reinforcement learning. The learning of the neural network may be a process in which the neural network applies knowledge for performing a specific operation to the neural network.

The neural network may be learned in a direction to minimize errors of an output. The learning of the neural network is a process of repeatedly inputting training data into the neural network and calculating the output of the neural network for the training data and the error of a target and back-propagating the errors of the neural network from the output layer of the neural network toward the input layer in a direction to reduce the errors to update the weight of each node of the neural network. In the case of the supervised learning, the training data labeled with a correct answer is used for each training data (i.e., the labeled training data) and in the case of the unsupervised learning, the correct answer may not be labeled in each training data. That is, for example, the training data in the case of the supervised learning related to the data classification may be data in which category is labeled in each training data. The labeled training data is input to the neural network, and the error may be calculated by comparing the output (category) of the neural network with the label of the training data. As another example, in the case of the unsupervised learning related to the data classification, the training data as the input is compared with the output of the neural network to calculate the error. The calculated error is back-propagated in a reverse direction (i.e., a direction from the output layer toward the input layer) in the neural network and connection weights of respective nodes of each layer of the neural network may be updated according to the back propagation. A variation amount of the updated connection weight of each node may be determined according to a learning rate. Calculation of the neural network for the input data and the back-propagation of the error may constitute a learning cycle (epoch). The learning rate may be applied differently according to the number of repetition times of the learning cycle of the neural network. For example, in an initial stage of the learning of the neural network, the neural network ensures a certain level of performance quickly by using a high learning rate, thereby increasing efficiency and uses a low learning rate in a latter stage of the learning, thereby increasing accuracy.

In learning of the neural network, the training data may be generally a subset of actual data (i.e., data to be processed using the learned neural network), and as a result, there may be a learning cycle in which errors for the training data decrease, but the errors for the actual data increase. Overfitting is a phenomenon in which the errors for the actual data increase due to excessive learning of the training data. For example, a phenomenon in which the neural network that learns a cat by showing a yellow cat sees a cat other than the yellow cat and does not recognize the corresponding cat as the cat may be a kind of overfitting. The overfitting may act as a cause which increases the error of the machine learning algorithm. Various optimization methods may be used in order to prevent the overfitting. In order to prevent the overfitting, a method such as increasing the training data, regularization, dropout of omitting a part of the node of the network in the process of learning, utilization of a batch normalization layer, etc., may be applied.

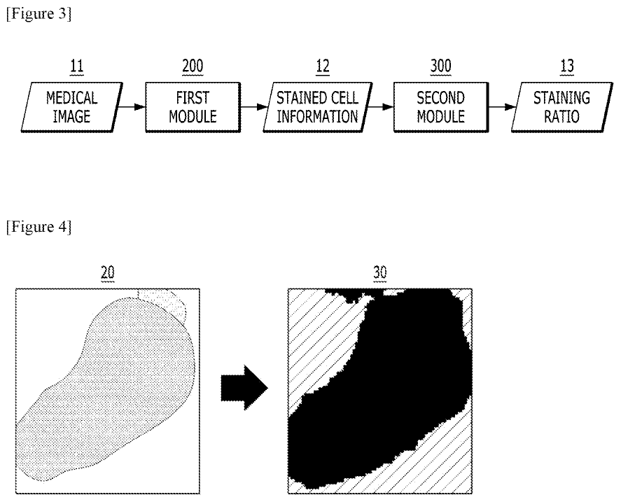

FIG. 3 is a block diagram illustrating a process of analyzing the medical image of the computing device according to an embodiment of the present disclosure.

Referring to FIG. 3 the processor 110 of the computing device 100 according to an embodiment of the present disclosure may generate stained cell information 12 present in a medical image 11 by using a pre-trained neural network model. The processor 110 may include a first module 200 that receives the medical image 11, and estimates the position of the stained cell present in the medical image 11. The first module 200 inputs at least a part of the medical image 11 into the neural network model to generate position information of the stained cell as stained cell information 12. For example, the first module 200 may estimate positions of respective cells stained with the DAB solution based on a medical image in which cells stained with the DAB solution are expressed by using the neural network model. Since the cell in which ki-67 is expressed is stained with a brown color through the DAB solution, the first module 200 inputs a pathology slide image in which the cells stained with the brown color are expressed into the neural network model to generate position information for each of the cells stained with the brown color.

According to an embodiment of the present disclosure, in order to obtain the stained cell information 12, the first module 200 inputs at least a part of the medical image 11 into the neural network model to generate a bounding box including the stained cell. Further, the first module 200 may generate the bounding box through the neural network model and at the same time, obtain coordinate values of the bounding box as position information of the stained cell. For example, the first module 200 may obtain a bounding box surrounding respective stained cells stained with the DAB solution based on a medical image in which cells stained with the DAB solution are expressed by using the neural network model. In this case, the bounding box may be understood as a virtual multiangled shape including the stained cells. Further, the first module 200 may obtain the bounding box of each stained cell and a coordinate value of the bounding box as the position information of each stained cell by using the neural network model. That is, the neural network model of the first module 200 receives the pathology slide image in which the stained cells are expressed to generate the bounding box for each stained cell and estimate the coordinate value of the bounding box. Conversely, the neural network model of the first module 200 receives the pathology slide image in which the stained cells are expressed to estimate the coordinate value of each of the stained cells, and generate a bounding box individually including the stained cells by using each coordinate value.

Meanwhile, the neural network model used to estimate the position of the stained cell in the first module 200 may also be a neural network model for segmenting a partial region of an image, and may also be a neural network model for detecting an object present in the image. For example, the neural network model may be a YOLOv3-based model for detecting the object present in the image. The neural network model may receive a pathology slide image as an input and output the coordinate value of the bounding box of the stained cell. When the neural network model is a model that performs classification (multi-classification) for multiple classes, the neural network model may detect multiple bounding boxes for the stained cells and calculate a score for each box. The neural network model may select the bounding box by comparing the score of each box with a predetermined threshold. The neural network model may determine the selected bounding box as the bounding box of the stained cell and output the coordinate value of the corresponding bounding box. Meanwhile, the neural network model of the present disclosure is not limited to the above-described YOLOv3 That is, various models capable of estimating the position of the stained cell based on the medical image may be applied as the neural network model of the present disclosure within a range understandable to those skilled in the art.

The neural network model used for estimating the position of the stained cell in the first module 200 may be pre-trained based on whether cells expressed by staining are positive or negative and a medical image in which the bounding box including cells is labeled. For example, the training data of the neural network model may include data in which whether each cell is DAB positive or DAB negative is labeled based on DAB staining by a domain expert (pathologist, etc.), and data in which the bounding box containing each cell is labeled. The neural network model can be trained to estimate the bounding box of the stained cell and the coordinate value of the bounding box based on the above-described training data. Since not only the DAB staining but also other immunohistochemistry may be all applied to the training data of the neural network model, the neural network model may be trained to estimate the bounding box of the stained cell present in the medical image and the coordinate value of the bounding box regardless of the type of immunohistochemistry.

Referring to FIG. 3, the processor 110 may calculate a staining ratio 13, which is a ratio of stained cells occupying a specific region of the medical image 11, based on the stained cell information 12 acquired by the first module 200. The processor 110 may include a second module 300 that receives the stained cell information 12 and estimates the staining ratio 13 within the bounding box of the stained cell present in the medical image 11. The second module 300 may calculate the staining ratio 13 of the stained cells within the bounding box of the medical image corresponding to the position information of the stained cell calculated as the stained cell information 12. At this time, the bounding box as a partial region of the medical image may be a polygonal region surrounding the stained cell.

For example, the second module 300 may extract a bounding box surrounding the DAB-stained cell based on the position information of the DAB-stained cell generated through the first module 200. At this time, the second module 300 may directly define the bounding box surrounding the DAB stained cell based on the coordinate value of the DAB stained cell calculated through the neural network model of the first module 200, and then extract the defined bounding box. The second module 300 may also define extract the bounding box of the DAB stained cell calculated through the neural network model of the first module 200 as the bonding box surrounding the DAB stained cell as it is. When extracting the bounding box of the DAB stained cell, the second module 300 may calculate a staining ratio which is a ration occupied by the DAB stained cell in the bounding box. Since a cell expressing ki-67 are stained with the brown color through the DAB solution, the second module 300 may calculate a ratio of a region expressed in the brown color within the bounding box as the staining ratio of the DAB-stained cell. A prediction ratio calculated through the second module 300 may be effectively used for diagnosing a pathological state by measuring an expression degree of a specific protein such as Ki-67.

According to an embodiment of the present disclosure, in order to obtain the staining ratio 13, the second module 300 may generate a binary image for the bounding box based on the staining intensity of the medical image. The second module 300 may generate the binary image of the bounding box surrounding the stained cells by comparing a staining expression intensity of the stained cells present in the medical image with a first threshold. At this time, the first threshold may be a staining expression intensity that serves as a reference for classifying the stained cell into the positive cell.

For example, the second module 300 determines the staining intensity of the entire bounding box based on the first threshold to distinguish the positive region of the cell expressed by staining and the remaining region. The second module 300 compares the staining intensity of the bounding box with the first threshold by the unit of a pixel to determine that pixels whose staining intensity is equal to or more than the first threshold are pixels in the positive region, and pixels whose staining intensity is equal to or less than the first threshold are pixels in the remaining region. Specifically, in the case of the DAB staining, the second module 300 may determine a pixel whose brown color intensity expressed in a pixel constituting the bounding box is equal to or more than the first threshold as a pixel of a cell corresponding to DAB positive. Conversely, the second module 300 may determine a pixel whose brown color intensity expressed in the pixel constituting the bounding box is equal to or less than the first threshold as a pixel of a cell corresponding to DAB negative or another tissue other than the stained cell. The second module 300 assigns a specific value, such as 1, to the pixel in the positive region (the pixel of the cell corresponding to DAB positive), and assigns no value to the pixel of the remaining region (the pixel of the cell corresponding to DAB negative or another tissue other than the stained cell) to generate the binary image of the bonding box.

The second module 300 may calculate the staining ratio 13 of the stained cells based on the binary image of the above-described bounding box. At this time, the staining ratio 13 may be understood as a ratio of an area of the positive region of cells expressed by staining within the bounding box to a total area of the bounding box. That is, since the staining ratio 13 is a value indicating how large the positive region of the cell expressed by staining occupies in the bounding box, the second module 300 may output, as the staining ratio 13, a ratio of an area of a region to which a specific value is assigned in the binary image to an area of a whole region of the binary image. Through the binary image, the bounding box is clearly divided into a positive region and the remaining region according to the first threshold, so the second module 300 may accurately calculate the staining ratio of positive cells expressed by staining within the bounding box by using the binary image.

For example, the second module 300 may calculate an area of the positive region (bounding box corresponding to DAB positive) to the specific value of 1 included in the binary image is assigned. The second module 300 may calculate an area of a whole region of the binary image, including both the positive region (bounding box corresponding to DAB positive) and the remaining region (bounding box corresponding to DAB negative or region of another tissue other than the stained cell). Based on the calculation results, the second module 300 may calculate the ratio of the area of the positive region (bounding box corresponding to DAB positive) to the area of the whole region of the binary image. Through such a calculation process, the second module 300 may measure an expression level of a specific protein and accurately calculate the ratio of positive cells expressed by staining, which is required for diagnosing the pathological state.

Meanwhile, the second module 300 may consider a size of the binary image of the bounding box in the process of calculating the staining ratio in order to increase the accuracy of calculating the staining ratio of the stained cells. In order to minimize the resources required to calculate the staining ratio to improve calculation efficiency and maintain the accuracy and reliability of the calculated values, the second module 300 may select a reference region for calculating the staining ratio in the binary image by considering the size of the binary image. The second module 300 may extract the reference region from the binary image based on a result of comparing the size of the binary image and a second threshold. The second module 300 may calculate a staining ratio of stained cells present in the reference region based on the reference region extracted from the binary image. At this time, the staining ratio may be a value of a ratio of an area of a positive region of a cell expressed by staining in the reference region to a total area of the reference region.

For example, when the size of the binary image is equal to or less than the second threshold, the second module 300 may determine the entire binary image as the reference region for calculating the staining ratio. Accordingly, when the size of the binary image is equal to or less than the second threshold, the second module 300 may calculate the staining ratio of the stained cells present in the binary image by using the binary image as it is. When the size of the binary image is equal to or more than the second threshold, the second module 300 may determine a partial region of the binary image, which occupies a predetermined ratio based on a center of the binary image as the reference region for calculating the staining ratio. The second module 300 may generate an image corresponding to the reference region by cropping the partial region of the binary image, which occupies the predetermined ratio based on the center of the binary image in the binary image. The second module 300 may calculate the staining ratio of the stained cells present in the reference region based on the image corresponding to the reference region. At this time, the second threshold may be 100 pixels by 100 pixels. Additionally, the predetermined ratio may be 50% of the whole region of the binary image. However, the specific values are only an example and may be changed variously within a range that may be selected by those skilled in the art. As such, when the reference region for calculating the staining ratio is calculated by considering the size of the binary image, the resources required to calculate the staining ratio are minimized while maintaining the accuracy and reliability of the calculated values, thereby improving the calculation efficiency.

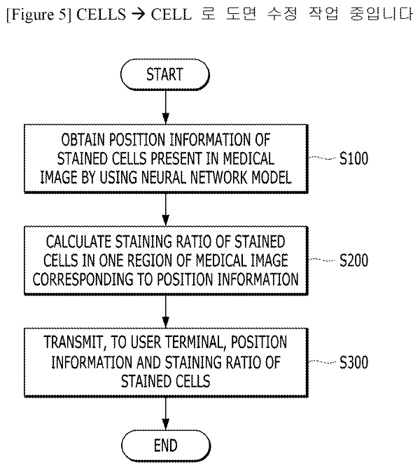

FIG. 4 is a conceptual view of a process of generating a binary image by the computing device according to an embodiment of the present disclosure.

Referring to FIG. 4 the processor 110 of the computing device 100 according to an embodiment of the present disclosure may generate a bounding box image 20 surrounding DAB stained cells present in the medical image by using a pre-trained neural network model. Since the bounding box image 20 is a minimum size image that includes all regions of one DAB-stained cell, the bounding box image 20 inevitably includes not only the DAB-stained cell but also some surrounding tissues of the DAB-stained cell. The bounding box image 20 in FIG. 4 is a square image, but the bounding box image 20 may be another multi-angled image such as a pentagonal image, or a circular image depending on setting by the user.

In order to accurately and easily calculate the staining ratio of the DAB-stained cells present in the bounding box image 20, the processor 110 may convert the bounding box image 20 into a binary image 30. In order to convert the bounding box image 20 into the binary image 30, the processor 110 may binary-process the bounding box image 20 by comparing staining intensities of pixels in the bounding box image 20 with a first threshold corresponding to a minimum reference intensity to be classified into a DAB positive cell. That is, the processor 110 may generate a binary image 30 based on pixels corresponding to positive cells expressed by DAB staining in the bounding box image 20. In the binary image 30, a black region indicates the DAB positive region and a hatched region indicates the DAB negative region or a surrounding tissue region other than the DAB stained bounding box. The processor 110 easily distinguishes the DAB positive region through the binary image 30 to quickly and accurately calculate a ratio of an area of the DAB positive region to a whole region of the binary image 30.

FIG. 5 is a flowchart illustrating a process of analyzing the medical image of the computing device according to an embodiment of the present disclosure.

Referring to FIG. 5, in step S100, a computing device 100 according to an embodiment of the present disclosure may receive the medical image through communication with a user terminal or PACS. At this time, the medical image may also be a whole slide image (WSI) including cells stained through immunohistochemistry (e.g., DAB staining), and may also be a partial image of the whole slide image (WSI) designated as a region of interest by the user. In step S100, when the medical image is received through communication with the user terminal or PACS, the computing device 100 may obtain position information of stained cells present in the medical image using a neural network model. For example, the computing device 100 may obtain coordinate values of the stained cell by inputting the whole slide image (WSI) or a partial image of the whole slide image (WSI) corresponding to the region of interest into the neural network model. At this time, the computing device 100 may obtain the bounding box containing the stained cells along with the coordinate values of the stained cell through the neural network model. At this time, the bounding box may be a polygonal or circular image of a minimum size adjacent to outlines of the stained cells.

In step S200, the computing device 100 may extract an image of a predetermined region including the stained cells from the medical image based on the position information of the stained cell calculated in step S100. For example, the computing device 100 may estimate a bounding box corresponding to the coordinate value of the stained cell based on the coordinate value of the stained cell, and extract the image corresponding to the bounding box from the medical image. At this time, when the bounding box is obtained along with the coordinate value of the stained cell in step S100, the computing device 100 may not extract the image of the predetermined region including the stained cell from the medical image, but use the bounding box obtained in step S100 as it is. In step S200, the computing device 100 may calculate the staining ratio of stained cells within one region of the medical image corresponding to the position information calculated in step S100. At this time, one region of the medical image may be a bounding box region previously extracted from the medical image. The computing device 100 may compare the staining intensity and a threshold, and calculate a staining ratio indicating how large the positive region of cells expressed by staining occupies based on the whole region of the bounding box.

In step S300, the computing device 100 may transmit the position information of the stained cells calculated through step S100 and the staining ratio calculated through step S200 to the user terminal. The computing device 100 may transmit, to the user terminal, only information requisitely utilized for pathological diagnosis, rather than the analyzed image itself. Through this, the computing device 100 prevents an unnecessarily large amount of resources from being required in the process of transmitting and receiving data with the user terminal and minimizes costs to enable efficient data communication. The position information of the stained cells calculated by the computing device 100 in step S100 and the staining ratio of the stained cells calculated by the computing device 200 in step S200 may be effectively used in a calculation process of counting the stained cells for the pathological diagnosis. In addition, the position information of the stained cells calculated by the computing device 100 in step S100 and the staining ratio of the stained cells calculated by the computing device 100 in step S200 may be effectively used for visualizing the image for the stained cells and the counting result of the stained cells through the user terminal.

In the meantime, according to an embodiment of the present disclosure, a computer readable medium storing a data structure is disclosed.

The data structure may refer to organization, management, and storage of data that enable efficient access and modification of data. The data structure may refer to organization of data for solving a specific problem (for example, data search, data storage, and data modification in the shortest time). The data structure may also be defined with a physical or logical relationship between the data elements designed to support a specific data processing function. A logical relationship between data elements may include a connection relationship between user defined data elements. A physical relationship between data elements may include an actual relationship between the data elements physically stored in a computer readable storage medium (for example, a permanent storage device). In particular, the data structure may include a set of data, a relationship between data, and a function or a command applicable to data. Through the effectively designed data structure, the computing device may perform a calculation while minimally using resources of the computing device. In particular, the computing device may improve efficiency of calculation, reading, insertion, deletion, comparison, exchange, and search through the effectively designed data structure.

The data structure may be divided into a linear data structure and a non-linear data structure according to the form of the data structure. The linear data structure may be the structure in which only one data is connected after one data. The linear data structure may include a list, a stack, a queue, and a deque. The list may mean a series of dataset in which order exists internally. The list may include a linked list. The linked list may have a data structure in which data is connected in a method in which each data has a pointer and is linked in a single line. In the linked list, the pointer may include information about the connection with the next or previous data. The linked list may be expressed as a single linked list, a double linked list, and a circular linked list according to the form. The stack may have a data listing structure with limited access to data. The stack may have a linear data structure that may process (for example, insert or delete) data only at one end of the data structure. The data stored in the stack may have a data structure (Last In First Out, LIFO) in which the later the data enters, the sooner the data comes out. The queue is a data listing structure with limited access to data, and may have a data structure (First In First Out, FIFO) in which the later the data is stored, the later the data comes out, unlike the stack. The deque may have a data structure that may process data at both ends of the data structure.

The non-linear data structure may be the structure in which the plurality of data is connected after one data. The non-linear data structure may include a graph data structure. The graph data structure may be defined with a vertex and an edge, and the edge may include a line connecting two different vertexes. The graph data structure may include a tree data structure. The tree data structure may be the data structure in which a path connecting two different vertexes among the plurality of vertexes included in the tree is one. That is, the tree data structure may be the data structure in which a loop is not formed in the graph data structure.

Throughout the present specification, a calculation model, a nerve network, the network function, and the neural network may be used with the same meaning. Hereinafter, the terms of the calculation model, the nerve network, the network function, and the neural network are unified and described with a neural network. The data structure may include a neural network. Further, the data structure including the neural network may be stored in a computer readable medium. The data structure including the neural network may also include preprocessed data for processing by the neural network, data input to the neural network, a weight of the neural network, a hyper-parameter of the neural network, data obtained from the neural network, an active function associated with each node or layer of the neural network, and a loss function for training of the neural network. The data structure including the neural network may include predetermined configuration elements among the disclosed configurations. That is, the data structure including the neural network may include the entirety or a predetermined combination of pre-processed data for processing by neural network, data input to the neural network, a weight of the neural network, a hyper parameter of the neural network, data obtained from the neural network, an active function associated with each node or layer of the neural network, and a loss function for training the neural network. In addition to the foregoing configurations, the data structure including the neural network may include predetermined other information determining a characteristic of the neural network. Further, the data structure may include all type of data used or generated in a computation process of the neural network, and is not limited to the foregoing matter. The computer readable medium may include a computer readable recording medium and/or a computer readable transmission medium. The neural network may be formed of a set of interconnected calculation units which are generally referred to as “nodes.” The “nodes” may also be called “neurons.” The neural network consists of one or more nodes.

The data structure may include data input to the neural network. The data structure including the data input to the neural network may be stored in the computer readable medium. The data input to the neural network may include training data input in the training process of the neural network and/or input data input to the training completed neural network. The data input to the neural network may include data that has undergone pre-processing and/or data to be pre-processed. The pre-processing may include a data processing process for inputting data to the neural network. Accordingly, the data structure may include data to be pre-processed and data generated by the pre-processing. The foregoing data structure is merely an example, and the present disclosure is not limited thereto.

The data structure may include a weight of the neural network (in the present specification, weights and parameters may be used with the same meaning), Further, the data structure including the weight of the neural network may be stored in the computer readable medium. The neural network may include a plurality of weights. The weight is variable, and in order for the neural network to perform a desired function, the weight may be varied by a user or an algorithm. For example, when one or more input nodes are connected to one output node by links, respectively, the output node may determine a data value output from the output node based on values input to the input nodes connected to the output node and the weight set in the link corresponding to each of the input nodes. The foregoing data structure is merely an example, and the present disclosure is not limited thereto.

For a non-limited example, the weight may include a weight varied in the neural network training process and/or the weight when the training of the neural network is completed. The weight varied in the neural network training process may include a weight at a time at which a training cycle starts and/or a weight varied during a training cycle. The weight when the training of the neural network is completed may include a weight of the neural network completing the training cycle. Accordingly, the data structure including the weight of the neural network may include the data structure including the weight varied in the neural network training process and/or the weight when the training of the neural network is completed. Accordingly, it is assumed that the weight and/or a combination of the respective weights are included in the data structure including the weight of the neural network. The foregoing data structure is merely an example, and the present disclosure is not limited thereto.

The data structure including the weight of the neural network may be stored in the computer readable storage medium (for example, a memory and a hard disk) after undergoing a serialization process. The serialization may be the process of storing the data structure in the same or different computing devices and converting the data structure into a form that may be reconstructed and used later. The computing device may serialize the data structure and transceive the data through a network. The serialized data structure including the weight of the neural network may be reconstructed in the same or different computing devices through deserialization. The data structure including the weight of the neural network is not limited to the serialization. Further, the data structure including the weight of the neural network may include a data structure (for example, in the non-linear data structure, B-Tree, Trie, m-way search tree, AVL tree, and Red-Black Tree) for improving efficiency of the calculation while minimally using the resources of the computing device. The foregoing matter is merely an example, and the present disclosure is not limited thereto.

The data structure may include a hyper-parameter of the neural network. The data structure including the hyper-parameter of the neural network may be stored in the computer readable medium. The hyper-parameter may be a variable varied by a user. The hyper-parameter may include, for example, a learning rate, a cost function, the number of times of repetition of the training cycle, weight initialization (for example, setting of a range of a weight value to be weight-initialized), and the number of hidden units (for example, the number of hidden layers and the number of nodes of the hidden layer). The foregoing data structure is merely an example, and the present disclosure is not limited thereto.

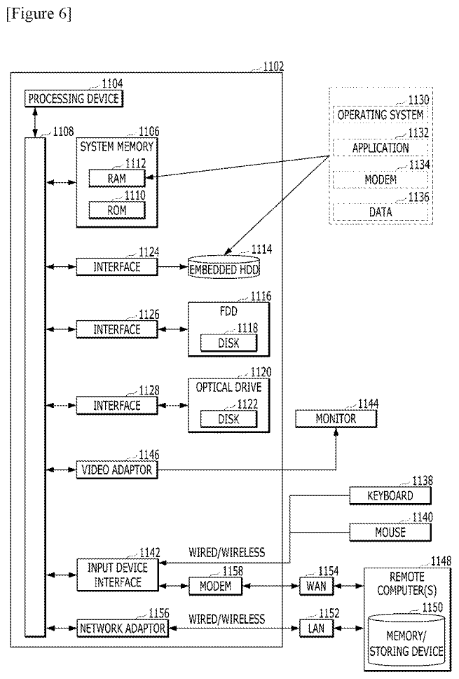

FIG. 6 is a simple and general schematic diagram illustrating an example of a computing environment in which the embodiments of the present disclosure are implementable.

The present disclosure has been described as being generally implementable by the computing device, but those skilled in the art will appreciate well that the present disclosure is combined with computer executable commands and/or other program modules executable in one or more computers and/or be implemented by a combination of hardware and software.

In general, a program module includes a routine, a program, a component, a data structure, and the like performing a specific task or implementing a specific abstract data form. Further, those skilled in the art will well appreciate that the method of the present disclosure may be carried out by a personal computer, a hand-held computing device, a microprocessor-based or programmable home appliance (each of which may be connected with one or more relevant devices and be operated), and other computer system configurations, as well as a single-processor or multiprocessor computer system, a mini computer, and a main frame computer.

The embodiments of the present disclosure may be carried out in a distribution computing environment, in which certain tasks are performed by remote processing devices connected through a communication network. In the distribution computing environment, a program module may be located in both a local memory storage device and a remote memory storage device.

The computer generally includes various computer readable media. The computer accessible medium may be any type of computer readable medium, and the computer readable medium includes volatile and non-volatile media, transitory and non-transitory media, and portable and non-portable media. As a non-limited example, the computer readable medium may include a computer readable storage medium and a computer readable transport medium. The computer readable storage medium includes volatile and non-volatile media, transitory and non-transitory media, and portable and non-portable media constructed by a predetermined method or technology, which stores information, such as a computer readable command, a data structure, a program module, or other data. The computer readable storage medium includes a RAM, a Read Only Memory (ROM), an Electrically Erasable and Programmable ROM (EEPROM), a flash memory, or other memory technologies, a Compact Disc (CD)-ROM, a Digital Video Disk (DVD), or other optical disk storage devices, a magnetic cassette, a magnetic tape, a magnetic disk storage device, or other magnetic storage device, or other predetermined media, which are accessible by a computer and are used for storing desired information, but is not limited thereto.

The computer readable transport medium generally implements a computer readable command, a data structure, a program module, or other data in a modulated data signal, such as a carrier wave or other transport mechanisms, and includes all of the information transport media. The modulated data signal means a signal, of which one or more of the characteristics are set or changed so as to encode information within the signal. As a non-limited example, the computer readable transport medium includes a wired medium, such as a wired network or a direct-wired connection, and a wireless medium, such as sound, Radio Frequency (RF), infrared rays, and other wireless media. A combination of the predetermined media among the foregoing media is also included in a range of the computer readable transport medium.

An illustrative environment 1100 including a computer 1102 and implementing several aspects of the present disclosure is illustrated, and the computer 1102 includes a processing device 1104, a system memory 1106, and a system bus 1108. The system bus 1108 connects system components including the system memory 1106 (not limited) to the processing device 1104. The processing device 1104 may be a predetermined processor among various commonly used processors. A dual processor and other multi-processor architectures may also be used as the processing device 1104.

The system bus 1108 may be a predetermined one among several types of bus structure, which may be additionally connectable to a local bus using a predetermined one among a memory bus, a peripheral device bus, and various common bus architectures. The system memory 1106 includes a ROM 1110, and a RAM 1112. A basic input/output system (BIOS) is stored in a non-volatile memory 1110, such as a ROM, an EPROM, and an EEPROM, and the BIOS includes a basic routing helping a transport of information among the constituent elements within the computer 1102 at a time, such as starting. The RAM 1112 may also include a high-rate RAM, such as a static RAM, for caching data.

The computer 1102 also includes an embedded hard disk drive (HDD) 1114 (for example, enhanced integrated drive electronics (EIDE) and serial advanced technology attachment (SATA))—the embedded HDD 1114 being configured for exterior mounted usage within a proper chassis (not illustrated)—a magnetic floppy disk drive (FDD) 1116 (for example, which is for reading data from a portable diskette 1118 or recording data in the portable diskette 1118), and an optical disk drive 1120 (for example, which is for reading a CD-ROM disk 1122, or reading data from other high-capacity optical media, such as a DVD, or recording data in the high-capacity optical media). A hard disk drive 1114, a magnetic disk drive 1116, and an optical disk drive 1120 may be connected to a system bus 1108 by a hard disk drive interface 1124, a magnetic disk drive interface 1126, and an optical drive interface 1128, respectively. An interface 1124 for implementing an outer mounted drive includes, for example, at least one of or both a universal serial bus (USB) and the Institute of Electrical and Electronics Engineers (IEEE) 1394 interface technology.

The drives and the computer readable media associated with the drives provide non-volatile storage of data, data structures, computer executable commands, and the like. In the case of the computer 1102, the drive and the medium correspond to the storage of random data in an appropriate digital form. In the description of the computer readable media, the HDD, the portable magnetic disk, and the portable optical media, such as a CD, or a DVD, are mentioned, but those skilled in the art will well appreciate that other types of computer readable media, such as a zip drive, a magnetic cassette, a flash memory card, and a cartridge, may also be used in the illustrative operation environment, and the predetermined medium may include computer executable commands for performing the methods of the present disclosure.

A plurality of program modules including an operation system 1130, one or more application programs 1132, other program modules 1134, and program data 1136 may be stored in the drive and the RAM 1112. An entirety or a part of the operation system, the application, the module, and/or data may also be cached in the RAM 1112. It will be well appreciated that the present disclosure may be implemented by several commercially usable operation systems or a combination of operation systems.

A user may input a command and information to the computer 1102 through one or more wired/wireless input devices, for example, a keyboard 1138 and a pointing device, such as a mouse 1140. Other input devices (not illustrated) may be a microphone, an IR remote controller, a joystick, a game pad, a stylus pen, a touch screen, and the like. The foregoing and other input devices are frequently connected to the processing device 1104 through an input device interface 1142 connected to the system bus 1108, but may be connected by other interfaces, such as a parallel port, an IEEE 1394 serial port, a game port, a USB port, an IR interface, and other interfaces.

A monitor 1144 or other types of display devices are also connected to the system bus 1108 through an interface, such as a video adaptor 1146. In addition to the monitor 1144, the computer generally includes other peripheral output devices (not illustrated), such as a speaker and a printer.

The computer 1102 may be operated in a networked environment by using a logical connection to one or more remote computers, such as remote computer(s) 1148, through wired and/or wireless communication. The remote computer(s) 1148 may be a work station, a computing device computer, a router, a personal computer, a portable computer, a microprocessor-based entertainment device, a peer device, and other general network nodes, and generally includes some or an entirety of the constituent elements described for the computer 1102, but only a memory storage device 1150 is illustrated for simplicity. The illustrated logical connection includes a wired/wireless connection to a local area network (LAN) 1152 and/or a larger network, for example, a wide area network (WAN) 1154. The LAN and WAN networking environments are general in an office and a company, and make an enterprise-wide computer network, such as an Intranet, easy, and all of the LAN and WAN networking environments may be connected to a worldwide computer network, for example, the Internet.

When the computer 1102 is used in the LAN networking environment, the computer 1102 is connected to the local network 1152 through a wired and/or wireless communication network interface or an adaptor 1156. The adaptor 1156 may make wired or wireless communication to the LAN 1152 easy, and the LAN 1152 also includes a wireless access point installed therein for the communication with the wireless adaptor 1156. When the computer 1102 is used in the WAN networking environment, the computer 1102 may include a modem 1158, is connected to a communication computing device on a WAN 1154, or includes other means setting communication through the WAN 1154 via the Internet. The modem 1158, which may be an embedded or outer-mounted and wired or wireless device, is connected to the system bus 1108 through a serial port interface 1142. In the networked environment, the program modules described for the computer 1102 or some of the program modules may be stored in a remote memory/storage device 1150. The illustrated network connection is illustrative, and those skilled in the art will appreciate well that other means setting a communication link between the computers may be used.

The computer 1102 performs an operation of communicating with a predetermined wireless device or entity, for example, a printer, a scanner, a desktop and/or portable computer, a portable data assistant (PDA), a communication satellite, predetermined equipment or place related to a wirelessly detectable tag, and a telephone, which is disposed by wireless communication and is operated. The operation includes a wireless fidelity (Wi-Fi) and Bluetooth wireless technology at least. Accordingly, the communication may have a pre-defined structure, such as a network in the related art, or may be simply ad hoc communication between at least two devices.

The Wi-Fi enables a connection to the Internet and the like even without a wire. The Wi-Fi is a wireless technology, such as a cellular phone, which enables the device, for example, the computer, to transmit and receive data indoors and outdoors, that is, in any place within a communication range of a base station. A Wi-Fi network uses a wireless technology, which is called IEEE 802.11 (a, b, g, etc.) for providing a safe, reliable, and high-rate wireless connection. The Wi-Fi may be used for connecting the computer to the computer, the Internet, and the wired network (IEEE 802.3 or Ethernet is used). The Wi-Fi network may be operated at, for example, a data rate of 11 Mbps (802.11a) or 54 Mbps (802.11b) in an unauthorized 2.4 and 5 GHz wireless band, or may be operated in a product including both bands (dual bands).

Those skilled in the art may appreciate that information and signals may be expressed by using predetermined various different technologies and techniques. For example, data, indications, commands, information, signals, bits, symbols, and chips referable in the foregoing description may be expressed with voltages, currents, electromagnetic waves, magnetic fields or particles, optical fields or particles, or a predetermined combination thereof.

Those skilled in the art will appreciate that the various illustrative logical blocks, modules, processors, means, circuits, and algorithm operations described in relationship to the embodiments disclosed herein may be implemented by electronic hardware (for convenience, called “software” herein), various forms of program or design code, or a combination thereof. In order to clearly describe compatibility of the hardware and the software, various illustrative components, blocks, modules, circuits, and operations are generally illustrated above in relation to the functions of the hardware and the software. Whether the function is implemented as hardware or software depends on design limits given to a specific application or an entire system. Those skilled in the art may perform the function described by various schemes for each specific application, but it shall not be construed that the determinations of the performance depart from the scope of the present disclosure.

Various embodiments presented herein may be implemented by a method, a device, or a manufactured article using a standard programming and/or engineering technology. A term “manufactured article” includes a computer program, a carrier, or a medium accessible from a predetermined computer-readable storage device. For example, the computer-readable storage medium includes a magnetic storage device (for example, a hard disk, a floppy disk, and a magnetic strip), an optical disk (for example, a CD and a DVD), a smart card, and a flash memory device (for example, an EEPROM, a card, a stick, and a key drive), but is not limited thereto. Further, various storage media presented herein include one or more devices and/or other machine-readable media for storing information.

It shall be understood that a specific order or a hierarchical structure of the operations included in the presented processes is an example of illustrative accesses. It shall be understood that a specific order or a hierarchical structure of the operations included in the processes may be rearranged within the scope of the present disclosure based on design priorities. The accompanying method claims provide various operations of elements in a sample order, but it does not mean that the claims are limited to the presented specific order or hierarchical structure.

The description of the presented embodiments is provided so as for those skilled in the art to use or carry out the present disclosure. Various modifications of the embodiments may be apparent to those skilled in the art, and general principles defined herein may be applied to other embodiments without departing from the scope of the present disclosure. Accordingly, the present disclosure is not limited to the embodiments suggested herein, and shall be interpreted within the broadest meaning range consistent to the principles and new characteristics presented herein.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for analyzing a medical image based on deep learning, is the method performed by a computing device including at least one processor, the method comprising:
obtaining position information of a stained cell present in a medical image by using a pre-trained neural network model; and
calculating a staining ratio of the stained cell in a bounding box including the stained cell corresponding to the position information.

2. The method of claim 1, wherein the obtaining of the position information of the stained cell present in the medical image includes:
obtaining the bounding box including the stained cell and a coordinate value of the bounding box by inputting the medical image into the neural network model.

3. The method of claim 1, wherein the neural network model is pre-trained based on whether a cell expressed by staining is positive or negative and a medical image in which the bounding box including the cell is labeled.

4. The method of claim 1, wherein the staining ratio of the stained cell is a ratio of an area of a positive region of cell expressed by staining in the bounding box to a total area of the bounding box.

5. The method of claim 1, wherein the calculating of the staining ratio of the stained cell includes:
generating a binary image for the bounding box based on a staining intensity of the medical image, and
calculating the staining ratio of the stained cell based on the binary image.

6. The method of claim 5, wherein the binary image is generated based on a result of comparing the staining intensity of the bounding box and a first threshold, and
wherein the first threshold is a staining intensity which becomes a reference for classifying the stained cell into a positive cell.

7. The method of claim 5, wherein the calculating of the staining ratio of the stained cell based on the binary image includes:
extracting a reference region from the binary image based on a result of comparing a size of the binary image and a second threshold, and
calculating the staining ratio of the stained cell based on the extracted reference region.

8. The method of claim 7, wherein when the size of the binary image is equal to or less than the second threshold, the reference region is a whole region of the binary image.

9. The method of claim 7, wherein when the size of the binary image is more than the second threshold, the reference region is a partial region of the binary image based on a center of the binary image.

10. The method of claim 1, further comprising:
transmitting, to a user terminal, the position information of the stained cell obtained through the neural network model and the calculated staining ratio,
wherein the position information includes a coordinate value of the bounding box, and
wherein the staining ratio is a value of a ratio of an area of a positive region of a cell expressed by staining in the bounding box to a total area of the bounding box.

11. A computer program stored in a computer-readable storage medium, wherein the computer program cause one or more processors to execute following operations for analyzing a medical image based on deep learning when the computer program is executed by the one or more processors, the operations comprising:
an operation of obtaining position information of a stained cell present in a medical image by using a pre-trained neural network model; and
an operation of calculating a staining ratio of the stained cell in a bounding box including the stained cell corresponding to the position information.

12. A computing device for analyzing a medical image based on deep learning, comprising:
a processor including at least one core;
a memory including program codes executable in the processor; and
a network unit receiving a medical image or transmitting a calculation result of the processor to a user terminal,
wherein the processor is configured to:
obtain position information of a stained cell present in a medical image by using a pre-trained neural network model, and
calculate a staining ratio of the stained cell in a bounding box including the stained cell corresponding to the position information.
